SEQUENCE LISTING (35 sequences)

SEQ ID NO: 1 - DNA, 561 bp, Brucella melitensis
  misc_feature (4)..(4): n = a, c, g, or t.
catngatggccaggngtcgantagtcctaaagtgtgatatcgatgtcattnaaagctatttccgtatgcccgacctgcnncagcgccccgaaggntggaaattctcgccgggcacagaaggtgatgcggaatttgcaagctgggtcgcgcgcaatgtcaccggggccaagcatcccgattatgccgtcgtcaccatctcgctgaagcctgtcggcgggcactcccggcgatgccagtgctgaacagatgaactggtgccgatgtggcgcaaacctgttccttcgatgaaatccgggtgagccacgagcagaacctcgtgctgccacatgtggccaaggccgatctgccggcagtctacgaccgcctgcaatcgggcggactggtgacaccccatgccggtctcatcaccgatatcattgcttgccccggtcttgattattgcgcgctggccaatgcacgctccattcccgttgcgcagcgcatttccgagcgcttcgccgatctggaccgtcagcttgaaatcggcgatctcaagatcaagatttccggctgcatcaatgc

SEQ ID NO: 2 - protein, 103 aa, Brucella melitensis
TGADVAQTCSFDEIRVSHEQNLVLPHVAKADLPAVYDRLQSGGLVTPHAGLITDIIACPGLDYCALANARSIPVAQRISERFADLDRQLEIGDLKIKISGCIN

SEQ ID NO: 3 - DNA, 393 bp, Brucella melitensis
  CDS (1)..(393)
gatatcgcgaangaaacggcngatgncatcctgctggaaangancctgatcgtgctggacgaaggcgtggtcacgnaccgcgagaccttcgggnatatgatgaactncctcatcatgtgccccagcttgaactttgcgaangtctgctacnggatggcggcaancgcattgatccagtgcnttacnatgcncntcattcaccaactgntccagaacctgatgtacnatatctcccaggtngagctgtcgaggaacaagatggacaaaggagattctggccaagccccgccgatgggatgccagaaacatgngcgcttcatgatctggacggctccgacctcgtcgatcatggacatnccacntttgcgctnantgncgtangcgttttcngcc

SEQ ID NO: 4 - protein, 131 aa, Brucella melitensis
  misc_feature (4)..(4): The ′Xaa′ at location 4 stands for Lys, or Asn.
DIAXETADXILLEXXLIVLDEGVVTXRETFGXMMNXLIMCPSLNFAXVCYXMAAXALIQCXXMXXIHQLXQNLMYXISQVELSRNKMDKGDSGQAPPMGCQKHXRFMIWTAPTSSIMDXPXLRXXXXAFXA

SEQ ID NO: 5 - DNA, 369 bp, Brucella melitensis
  CDS (2)..(367)
gatcggcacgggcgacaccaagagtgatgaactgcttttcatggcggatgtcatcgaaagggctggcggctcaccggtgatgatcgatgtcagcattntgggcaatccgccttatgagccggcttattccaagcatgatgtggctgaagccgccggaacgacggttcaggcgattatcgacagcggcgatgaacatagcgccatggccctgatggccgaaggggcgacggctctcgttcgtggcctttcgcaacgcggccaggtcgatggcatgattgcgcttggcggttcgcttggcacggaccttgcgctggatattgcggcgatcctgccgcttgtcgtgccgaaattcatcgtttcgaccattgc

SEQ ID NO: 6 - protein, 122 aa, Brucella melitensis
  misc_feature (33)..(33): The ′Xaa′ at location 33 stands for Met, Val, or Leu.
IGTGDTKSDELLFMADVIERAGGSPVMIDVSIXGNPPYEPAYSKHDVAEAAGTTVQAIIDSGDEHSAMALMAEGATALVRGLSQRGQVDGMIALGGSLGTDLALDIAAILPLVVPKFIVSTI

SEQ ID NO: 7 - DNA, 368 bp, Brucella melitensis
  CDS (3)..(308)
cgcttgccggaacggcgattggcctgaaggaacggaaccacaatatcaaaattgctctggcggacccgcatggcgcggcgcttcacgccttctatacgaccggcgaattgaaggcagaaggcgattcgatcaccgaaggcatcgggcaggggcgcatcaccgccaatcttgaaggcttcacccctgatttttcctaccagattcccgatgcggaggcgctcgatatcctgtttgccctcgtggaagaggaggggctttgcctcggcggctcgtccggcatcaacattgccggtgcaatccctgtctcttgatcagatctggccgcctagaatcactagtgaattcgcggccgcctgcaggtcgaccat

SEQ ID NO: 8 - protein, 102 aa, Brucella melitensis
LAGTAIGLKERNHNIKIALADPHGAALHAFYTTGELKAEGDSITEGIGQGRITANLEGFTPDFSYQIPDAEALDILFALVEEEGLCLGGSSGINIAGAIPVS

SEQ ID NO: 9 - DNA, 398 bp, Brucella melitensis
  CDS (2)..(238)
aaatccattcgcgttacgagcgcggcaggctgtctatgtcgtggatgcaagtcgtgcggtgggggtcgtgtcaaacctcctgtcgccggagggcaagcaggcctatatcgacggtttacgcaacgaatatgccaaggtggctgctgcccatgcccgcaacgaggctgaaaaacagcgcctgccaattgcccgcgcgcgcgccaatccgcatcagctcgactgggaaaactacgagccggtgaagccgaccttcaccggaacgaaggtttttgaaacctatgatctggcgaaatcgcgcgctatatcgactggaccccgttcttccagacctgggaactgaggggccgctatccggctattctcgaagatgaaaagcagggtgaggcggcgcgccagct

SEQ ID NO: 10 - protein, 79 aa, Brucella melitensis
NPFALRARQAVYVVDASRAVGVVSNLLSPEGKQAYIDGLRNEYAKVAAAHARNEAEKQRLPIARARANPHQLDWENYEP

SEQ ID NO: 11 - DNA, 103 bp, Brucella melitensis
  CDS (3)..(101)
ggaccgaagccgactggcggcttttcaccacgcttgtgcggttcgatccggtctatgtcggccatttcaaatgcaatcttcgcaggctcgcagattatccaaa

SEQ ID NO: 12 - protein, 33 aa, Brucella melitensis
TEADWRLFTTLVRFDPVYVGHFKCNLRRLADYP

SEQ ID NO: 13 - DNA, 559 bp, Brucella melitensis
  CDS (1)..(414)
tttcagggtggcggctttggcggtgcggaagatattctgaacgatcttttcggcggtgcttttggcggcggtcgctctggcgcgcgccagcgcggcccgtccaagggggcggacctgtctgcatccattgatatttcgctttcgcaggccgtgggcgcggaaaaggtcgaggctatctttccaaacggcaagcatctgaaaataaaactgccaaaattcgtcgaggatggccagaccattcgcctcaaggggcagggtgaaccgctgatgggcggtacgccgggggacgcgctggtgacgatccgcttcaagccgcattcgcgtttccgcttggaaggccgcgacgttcatgtcgaccttccggtcagcattgacgatgcggttctgggcggcaagcangaaagtggaaactcttgatgggcgcatttcggtgaagattcccgcatggnccagtttcgacccgcgtaatgcgccttgaaggaaaaaggcctttcctttgaaanccgggnggnnaggggctaacctctatgnncatgtccggatcatgcttgccggaagg

SEQ ID NO: 14 - protein, 138 aa, Brucella melitensis
FQGGGFGGAEDILNDLFGGAFGGGRSGARQRGPSKGADLSASIDISLSQAVGAEKVEAIFPNGKHLKIKLPKFVEDGQTIRLKGQGEPLMGGTPGDALVTIRFKPHSRFRLEGRDVHVDLPVSIDDAVLGGKTESGNS

SEQ ID NO: 15 - DNA, 632 bp, Brucella melitensis
  CDS (3)..(536)
ctgatcaagagacaggccctgccacgatcgggcgggtcaatctctttgcttcgccagatggtgttttctgcaccgacaccgccggtatcgacgccatcaatatgcatgatccgcgtatatccgttgcgaccatacgcaatcacgcgcgtgtcgaagcggggcagatggttgcgacgatcaagatcattccctttgccgttccgggcgtcctgatcgaagaaattcgcgcagtcgtgcacggcagaccgcttttacaggtcgaaaaattccggaatatgcaggtcggcctcatccagtcgcgcctgccctcgatccgcgaaacggttctcgacaggacgctggagcttatgcacaagcgcgtaacccgcaatggcggcgcggtggtcacgcaagagcgcgtcgcacatcatgacgaggcgcttgcgacagccattgcaaaggttggcagcgcctgcgatctcatcgtcattttcagtgctttcggcggtggcccgatgaaccgacatcggtccgcgcgccatcaggcgcgccgggggtgagatcggtgaagtcgccgcngcgctcgcaatcggacgcctgctcgccggcaacgggatcgaacccggccctggcactgattttatacacaagtg

SEQ ID NO: 16 - protein, 178 aa, Brucella melitensis
DQETGPATIGRVNLFASPDGVFCTDTAGIDAINMHDPRISVATIRNHARVEAGQMVATIKIIPFAVPGVLIEEIRAVVHGRPLLQVEKFRNMQVGLIQSRLPSIRETVLDRTLELMHKRVTRNGGAVVTQERVAHHDEALATAIAKVGSACDLIVIFSAFGGGPMNRHRSARHQARRG

SEQ ID NO: 17 - DNA, 120 bp, Brucella melitensis
  CDS (3)..(119)
atatccgcctcatcaaacccgaccgggcgatctacgatcatcacgtcgcctcgttcggcctcgatccggcagcaacgctcttcatcgatgacacgatgcacaatgtggaaggtgcgaagc

SEQ ID NO: 18 - protein, 39 aa, Brucella melitensis
IRLIKPDRAIYDHHVASFGLDPAATLFIDDTMHNVEGAK

SEQ ID NO: 19 - DNA, 641 bp, Brucella melitensis
  CDS (3)..(641)
tgctgggcgtgcggcggcgcggaaatttcgtcgacctgcaggtgctcgacaccggcatcggcattgcgccacaaaaactgaagctcgtcttccgggagttcacgcggctcaacgaaggcatgcgggaagccgaagggctggggcttggcctttccatcgtggaccgcatcgcgcgggtgctttcgctgccgctgtcgcttgcctcaacacccggcaaaggtacggtgttttcgctgcgcattccggtttcaggcgaggcgccgccagccgaagaggttaaaaaccgtcgcgggcctcagcgcgcttccgagcttgacgggcttggcgtcctttgcatcgacaatgacgtcaatattctatgcggcatggaaacgctgctgagcggctggggctgcaacgtgaccacgcttcgcagcggcgcggagttgaaaaacttctgcgcaaccggcacggccgcgcctgccgtcatcattgccgattatcatctgatccacgaaaacggcctcgacatgatcggctttgcgcgcgaaagcttcaagaccgaaataccggccatcctgctaactgccgaccgctccaaggaggtgcgccagcgcgccgaggatgagaacgtcaccgtgctgcacaagccctgtctcttg

SEQ ID NO: 20 - protein, 213 aa, Brucella melitensis
LGVRRRGNFVDLQVLDTGIGIAPQKLKLVFREFTRLNEGMREAEGLGLGLSIVDRIARVLSLPLSLASTPGKGTVFSLRIPVSGEAPPAEEVKNRRGPQRASELDGLGVLCIDNDVNILCGMETLLSGWGCNVTTLRSGAELKNFCATGTAAPAVIIADYHLIHENGLDMIGFARESFKTEIPAILLTADRSKEVRQRAEDENVTVLHKPCLL

SEQ ID NO: 21 - DNA, 403 bp, Brucella melitensis
  CDS (1)..(267)
ggactgggggatatcgacctcgaccacccgaaagacccggaaatcgaaccgctcattgcgcggctgcgcaccctgacgccgcagcaaacccgcgttctgaccatgcttgccgaggggcttctcaacaaacagatcgcttttgagcttggcgtttcggaggcaacggtcaaggcgcatgtttccgccgtgctgcaaaagcttggcgtcgatagccgcacacaggccgtcattctcctttcgcgtatcggcagcgacgttctgggcgtttgagccagctcagctataggagctgtcggcctttgccgcttatcgtgtactgccttgcgtcgatgccccgtcctggcgtgcgtgatgaaaaagcgagagcatcgagcgcagcgccgctggccgcaagggcttgtgc

SEQ ID NO: 22 - protein, 89 aa, Brucella melitensis
GLGDIDLDHPKDPEIEPLIARLRTLTPQQTRVLTMLAEGLLNKQIAFELGVSEATVKAHVSAVLQKLGVDSRTQAVILLSRIGSDVLGV

SEQ ID NO: 23 - DNA, 536 bp, Brucella melitensis
  CDS (314)..(520)
ggtttcacggcgatcatcgttgcctttctgggacggcttaatccgctgggtgcgattgtggcgggccttgtgctggcgctttcctaccttggcggcgaggcggcacaggtggccattggcatatcggaaaagtccgcccgtgtgtttcagggcatgatcctgtttttcgtgcttgcttgcgatacgctcattctttatcgcatccgcattgtgatgaaccgccaggcggggaaggcctgatctcatggatttgacacaggcaatccttctcaccatcgctaccgcagcaacaccgctcctgattgcggccattggcgaactggtggtggaacgctccggcgttctcaatctcggcgtcgagggcatgagctgntgggcgcggtttccggcttcgccgtggcgcagatanccggctcggcctggctcggccttgttgccgccgttctggtcggggcgctgtttgcggcagtcttcggctttcttacgctcaccctcgtcaccaatcaggtggcgaccggccttgcacctgtctcttg

SEQ ID NO: 24 - protein, 69 aa, Brucella melitensis
  misc_feature (20)..(20): The ′Xaa′ at location 20 stands for a stop codon, Trp, or Cys.
GELVVERSGVLNLGVEGMSXWARFPASPWRRXPARPGSALLPPFWSGRCLRQSSAFLRSPSSPIRWRPA

SEQ ID NO: 25 - DNA, 190 bp, Brucella melitensis
  CDS (3)..(188)
gccttgcactcactattctgggcgtcggcgtatcggcattgcctggagaaagcttcgtcgggcttccgggcgcgcggctgaacccgatctatattccctatctgactgatcttccgctcatcgggcgctttctcttcggtcaggatcccatcttctatatatcccggccccgtgtactagtcgacgcgtg

SEQ ID NO: 26 - protein, 62 aa, Brucella melitensis
LALTILGVGVSALPGESFVGLPGARLNPIYIPYLTDLPLIGRFLFGQDPIFYISRPRVLVDA

SEQ ID NO: 27 - DNA, 360 bp, Brucella melitensis
  CDS (2)..(358)
ggcaccgcagatcgttttgactgtggttttcttcttctggccggcaagtcaggcaatttattantccttcatgcgggaagacgcattcgggctcaaatccaccttngtggaactggccaatttcaccgccgtcctttcggacccgaattatctccattccgttcaggtcacggtcgttttcaacgtcttgaccgccctgctcgccatgggtgtcgcgctgctgcttgcaaccgctgccgaccgcgtcattcgcggccagaccttctaccgtactcttctgatctggccctatgccgtggcgcctgctgtcgcgggcatgttgtggctgttcatgttcaatccggccatgggcacgtttgc

SEQ ID NO: 28 - protein, 119 aa, Brucella melitensis
  misc_feature (21)..(21): The ′Xaa′ at location 21 stands for a stop codon, or Tyr.
APQIVLTVVFFFWPASQAIYXSFMREDAFGLKSTXVELANFTAVLSDPNYLHSVQVTVVFNVLTALLAMGVALLLATAADRVIRGQTFYRTLLIWPYAVAPAVAGMLWLFMFNPAMGTF

SEQ ID NO: 29 - DNA, 375 bp, Brucella melitensis
  CDS (1)..(336)
aaaggcatcgcggaattcttcaatttcctctcgcagagcaagattcaggtcaagctgcatgaaaaatccggctatctgccggtgacgcttgcagcctatgaggaaaccaagaagtcggatttctatgaaaagaatccgggccgcgaaacgccgatcctgcaaatgatgggcaaggagccgacggaaaattccaagggtgtgcgcctcgtcaacctgccgcaggttcgcgacattctgaatgaagagtttgaagccatgctgggcggcaaacaggacgcgaagaccgcactcgacaacgccgtcaagcgcggcaacgcagcaatcgccgcagctcaataatccatcatatcgagacaatgccgccaaccctttccg

SEQ ID NO: 30 - protein, 112 aa, Brucella melitensis
KGIAEFFNFLSQSKIQVKLHEKSGYLPVTLAAYEETKKSDFYEKNPGRETPILQMMGKEPTENSKGVRLVNLPQVRDILNEEFEAMLGGKQDAKTALDNAVKRGNAAIAAAQ

SEQ ID NO: 31 - DNA, 249 bp, Brucella melitensis
  misc_feature (10)..(10): n = a, c, g, or t.
gttctggacnatgcnggagacgggggtccnagccgnggggcatccaccatcaccatgcagatggtgaagaacctgttnttgtggaatgggcgctcctatctgcgcaaagggctggaatttccgctggcgttgattctgatcaagagacaggtccagancgaggcctagctgatgccagtccaccccnccgtggctgcaaaattggccatcctccgccatcatcaccgaattgaccagaaccggcgcgat

SEQ ID NO: 32 - DNA, 333 bp, Brucella melitensis
gcgcagaggcactgcatgcgagcggttgcggatacccttgatcgcagtcagtgtggcaatggcttccccggcaggcaggtcgcacaggccgtgaagatcgagcgcaccgcctgccagtgcctcgcttaacgccagaagcgttctctgcttgggccgcgaaagcccggccaaacgccacgcctcctccccgcccgcgatataggcttccggcgtcagcggattgatcacctgtttcaaccgcgcccagatggcggcggcgctggcggtcgaaacctgctgcgcgacgacgatggaagccagactttcaaatccgggttcggaccggcgcagagg

SEQ ID NO: 33 - DNA, 99 bp, Brucella melitensis
cccagttcttgttcttgaaagtcacagatctcattgaatcatcccttttccgtatcgcgtctggtgatcgcgcagttcaattgacagggaatatagcgg

SEQ ID NO: 34 - DNA, 18 bp, Artificial Sequence
  primer sequence
tacctacaacctcaagct

SEQ ID NO: 35 - DNA, 18 bp, Artificial Sequence
  primer sequence
tacccattctaaccaagc

(19) United States
(12) Patent Application Publication
(10) Pub. No.: US 2006/0246086 A1
(43) Pub. Date: Nov. 2, 2006

(54) Virulence genes and proteins from Brucella melitensis, and their use

(76) Inventors: Pascal Lestrate, Namur (BE); Jean-Jacques Letesson, Namur (BE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/475,590

(22) Filed: Jun. 27, 2006

Related U.S. Application Data
(63) Continuation of application No. 10/129,066, filed on Aug. 13, 2002, now abandoned.

(30) Foreign Application Priority Data
Nov. 5, 1999 (GB) GB 9926340.2
Nov. 5, 1999 (GB) GB 9926341.0
Nov. 5, 1999 (GB) GB 9926342.8
Nov. 5, 1999 (GB) GB 9926343.6
Nov. 5, 1999 (GB) GB 9926344.4
Nov. 5, 1999 (GB) GB 9926345.1
Nov. 5, 1999 (GB) GB 9926346.9
Nov. 5, 1999 (GB) GB 9926347.7
Nov. 5, 1999 (GB) GB 9926348.5
Nov. 5, 1999 (GB) GB 9926350.1
Nov. 5, 1999 (GB) GB 9926351.9
Nov. 5, 1999 (GB) GB 9926356.8

(51) Int. Cl. A61K 39/00; A61K 39/38

(52) U.S. Cl. 424/184.1

(57) ABSTRACT

A series of genes from Brucella spp are shown to encode products which are implicated in virulence. The identification of these genes therefore allows attenuated microorganisms to be produced. Furthermore, the genes or their encoded products can be used in the manufacture of vaccines for therapeutic application.

### FIELD OF THE INVENTION

This invention relates to virulence genes and proteins, and their use. More particularly, it relates to genes and proteins/peptides obtained from Brucella species, and their use in therapy and in screening for drugs.

### BACKGROUND OF THE INVENTION

Members of the Gram-negative genus Brucella cause Brucellosis, an infectious disease of animals that is transmissible to humans. Brucellosis in humans is sometimes called Malta fever or undulant fever and can occur in adults, children or neonates. Brucellosis can result in either an acute or chronic disease and can manifest as a variety of symptoms including arthritis, enteric fever, meningitis, encephalitis, infective endocarditis, myocarditis and cutaneous lesions, as well as giving rise to heamatological abnormalities.

There are currently six species of Brucella identified; B. melitensis, B. suis, B. neotomae, B. ovis, B. canis and B. abortus. The principal hosts of B. melitensis are small ruminants (goats and sheep), although infections of cattle are also widespread.

B. melitensis is the most virulent species of the brucellae identified to date, based on the ability to cause infections in humans and cattle. B. melitensis infections in animals can result in abortion, sterility and decreased milk production in females, and epididymitis and orchitis in males. It would be desirable to provide means for treating or preventing conditions caused by Brucella species in animals and humans e.g. by immunisation.

### SUMMARY OF THE INVENTION

The present invention is based on the discovery of virulence genes in B. melitensis.

According to a first aspect of the invention, a peptide of the invention is encoded by an operon including any of the nucleotide sequences identified herein as SEQ ID NOS. 1, 3, 5, 7, 9, 11, 13, 15, 17, 19, 21, 23, 25, 27, 29, 31, 32 and 33 of B. melitensis, or a homologue thereof in a Gram-negative bacterium, or a functional fragment thereof, for therapeutic or diagnostic use.

The peptides may have many therapeutic uses for treating Brucella infections, including use in vaccines for prophylactic application.

According to a second aspect, a polynucleotide encoding a peptide defined above, may also be useful for therapy or diagnosis.

According to a third aspect, the genes that encode the peptides may be utilised to prepare attenuated microorganisms. The attenuated microorganisms will usually have a mutation that disrupts the expression of one or more of the genes identified herein, to provide a strain that lacks virulence. These microorganisms will also have use in therapy and diagnosis.

According to a fourth aspect, the peptides, genes and attenuated microorganisms according to the invention may be used in the treatment or prevention of a condition associated with infection by Brucella or Gram-negative bacteria.

### DESCRIPTION OF THE INVENTION

The present invention is based on the discovery of genes which encode peptides which are implicated in virulence. The peptides and genes of the invention are therefore very useful for the preparation of therapeutic agents to treat infection. It should be understood that references to therapy also include preventative treatments, e.g. vaccination. Furthermore, while the products of the invention are intended primarily for treatment of infections in human patients, veterinary applications are also considered to be within the scope of the invention.

The present invention is described with reference to Brucella melitensis. However, all the Brucella strains, and many other Gram-negative bacterial strains are likely to include related peptides or proteins having amino acid sequence homology to those identified herein. Organisms likely to contain the peptides include, but are not limited to, B. suis, B. neotomae, B. ovis, B. canis and B. abortus.

Preferably, the peptides that may be useful in the various aspects of the invention have greater than a 40% similarity with the peptides identified herein. More preferably, the peptides have greater than 60% sequence similarity. Most preferably, the peptides have greater than 80% sequence similarity, e.g. 95% similarity. With regard to the polynucleotide sequences identified herein, homologs that may be useful in the various aspects of the invention may have greater than 40% identity with the sequences identified herein. More preferably, the polynucleotide sequences have greater than 60% sequence identity. Most preferably, the polynucleotide sequences have greater than 80% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al, J. Molec. Biol., 1990; 215:403-410), the BLASTX program available from NCBI, and the Gap program from Genetics Computer Group, Madison Wis. The levels of similarity and identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to establish homologies in other microorganisms. In this way, it is possible to determine whether other microorganisms have similar peptides. Sequence homologies may be established by searching in existing databases, e.g. EMBL or GenBank.

Peptides or proteins according to the invention may be purified and isolated by methods known in the art. In particular, having identified the gene sequence, it will be possible to use recombinant techniques to express the genes in a suitable host. Active fragments and homologues can be identified and may be useful in therapy. For example, the peptides or their active fragments may be used as antigenic determinants in a vaccine, to elicit an immune response. They may also be used in the preparation of antibodies, for passive immunisation, or diagnostic applications. Suitable antibodies include monoclonal antibodies, or fragments thereof, including single chain Fv fragments. Methods for the preparation of antibodies will be apparent to those skilled in the art.

The preparation of vaccines based on attenuated microorganisms is known to those skilled in the art. Vaccine compositions can be formulated with suitable carriers or adjuvants, e.g. alum, as necessary or desired, to provide effective immunisation against infection. The preparation of vaccine formulations will be apparent to the skilled person. The attenuated microorganisms may be prepared with a mutation that disrupts the expression of any of the genes identified herein. The skilled person will be aware of methods for disrupting expression of particular genes. Techniques that may be used include insertional inactivation or gene deletion techniques. Attenuated microorganisms according to the invention may also comprise additional mutations in other genes, for example in a second gene identified herein or in a separate gene required for growth of the microorganism, e.g. an aro mutation. Attenuated microorganisms may also be used as carrier systems for heterologous antigens, therapeutic proteins or nucleic acids. In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include suitable promoter sequences. Alternatively, the gene that encodes the heterologous antigen or protein may be incorporated into the genome of the organism and the endogenous promoters used to control expression.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type of health of the subject etc.

In a separate embodiment, the products of the invention may be used in screening assays for the identification of potential antimicrobial drugs or for the detection for virulence. Routine screening assays are known to those skilled in the art, and can be adapted using the products of the invention in the appropriate way. For example, the products of the invention may be used as the target for a potential drug, with the ability of the drug to inactivate or bind to the target indicating its potential antimicrobial activity.

The products of the present invention were identified using the following procedure:

In summary, signature-tagged mutagenesis (STM) (Hensel et al., Science 1995; 269: 400-403) was used to identify genes required for the in vivo pathogenesis of Brucella.

Bacterial Strains, Plasmids and Matings.

B. melitensis 16M Nal^{r} (Verger et al., Brucella spp. Plasmid, 1993; 29:142-146) was used as the parental strain for all experiments. B. melitensis strains were grown on solid or liquid 2YT medium with appropriate antibiotics. The E. coli strains used in this study were: S17 λpir [recA thi pro hsdR⁻M^{+l RP}4::2-Tc::Mu::Km Tn7 lysogenised with λpir phage] (Miller and Mekalanos, J. Bacteriol., 1988; 170(6): 2575-2583), CC118_λ pir [Δ(are-leu) araD ΔlacX74 galE galK phoA20 thi-1 rpsE rpoB argE recA1 lysogenised with λ pir phage] (de Lorenzo et al., J. Bacteriol., 1990; 172:6568-6572), and TOP10 [F⁻ mcrA Δ(mrr-hsdRMS-mcrBC) Φ80lacZΔM15 ΔlacX14 recA1 deoR araD139 Δ(ara-leu)7697 galU galK rpsL (Str^{r}) endA1 nupG] (Invitrogen). E. coli strains were grown on Luria-Bertani (LB) medium with appropriate antibiotics. Antibiotics were used at the following concentration for E. coli and B. melitensis: Ampicillin (Amp), 50 μg/ml; Kanamycin (Kan), 50 μg/ml; Nalidixic acid (NaI), 25 μg/ml. A modified minimal medium was used. The plasmids used in this study were: pUTmini-Tn5 Km2 (Hensel et al., supra) and pCR TOPO 2.1 (Invitrogen).

The mutant bank was generated by transferring the mini-Tn5 transposon from E. coli Lambda pir strains where it is maintained on a plasmid to the B. melitensis chromosome. This is achieved by mating the E. coli strains containing signature-tagged pUTminiTn5Km2 with the B. melitensis parental strain resulting in transfer of the tagged transposon to the Brucella chromosome.

Matings were performed by mixing equal volumes (20 μl) of liquid cultures of E. coli S17 donor cells (OD 0.6) and the B. melitenisis 16M Nal^{r} recipient strain (overnight culture) on a 0.22 μm filter. The filter was left for one hour on a LB plate without antibiotics and then transferred onto a 2YT plate containing Kan and Nal. After three days incubation at 37° C., the exconjugates were replicated on 2YT NaI Kan and on 2YT NaI Amp. The Amp-resistant clones (about 4% of the clones) were discarded, the Amp-sensitive clones were transferred into 96 well plates.

Amplification and Labelling of DNA Tags from Signature Tagged Mutants.

A modified version of the protocol from Holden and Hensel (Methods in Microbiology, 1998; 27:359-370) was used for amplification and labelling of tags. Briefly, 10⁴ colonies of signature-tagged mutant bacteria from each input and output pool of 96 individual mutants were recovered from agar plates, resuspended in PBS, centrifuged and genomic DNA from the pelleted bacteria recovered by the CTAB method (Ausubel et al., Current Protocols in Molecular Biology, 1991). Tags were initially amplified by PCR from genomic DNA using the primers SEQ ID NO. 34 and 35.

The amplicons were purified and a fraction was used as target DNA in the second PCR including [32-P]-dCTP to radiolabel the tags. The presence or absence of the individual tags in the input and output pools was shown by hybridising the radiolabelled tags to DNA dot blots of the 96 signature tags, amplified by PCR from the 96 signature-tagged pUTminiTn5 Km2 plasmids.

Molecular Techniques.

DNA manipulation was performed following standard techniques (Ausubel et al., supra). Restriction enzymes were purchased from Roche and primers from Amersham Pharmacia.

Identification of Mini-Tn5 Insertion Sites.

Transposon insertion sites were amplified by arbitrary PCR(O'Toole and Kolter, Mol. Microbiol, 1998; 28:449-461) of genomic DNA isolated from the mini-TnS mutants. Arbitrary PCR was also performed on genomic DNA from the wild-type as negative control. The PCR products were cloned into pCR TOPO 2.1 (Invitrogen). The inserts were sequenced using the dye terminator method (Big Dye Terminator kit, Perkin Elmer) with an ABI 377 sequencer. Sequences were analysed by performing searches with the Blastx program (Altschul et al., Nucleic Acids Res, 1997; 25: 3389-3402) against the EMBL and GenBank databases.

Screening of the STM Library.

Mutants were each grown at 37° C. in 200 μl of 2YT in 96 well microtitre plates with appropriate antibiotics for 48 h. The bacteria were then pooled, centrifuged at 4000 r.p.m. for 10 min and resuspended in 2 ml of 0.9% NaCl. The bacterial suspension was then diluted to a final concentration of 5×10⁵ cfu in 100 μl of 0.9% NaCl. The number of bacteria was confirmed by plating dilutions on 2YT plates. The bacterial suspension was injected i.p. in five week-old female BALB/c mice. The remaining part of the suspension was plated onto media for DNA isolation. Five days after the infection, animals were sacrificed and the spleens removed aseptically. For recovery of bacteria, the spleens were homogenised in PBS 0.1% Triton X-100 (Roche) and dilutions were plated on 2YT. Plates containing approximately 10⁴ colonies were used for DNA extraction (the output pool). Signature tags were then amplified from the genomic DNA by a two-step PCR and stringent hybridisations performed as described above.

Competitive Index

Mutants identified by STM are attenuated in virulence. The level of attenuation was measured by determining the competitive index (CI).

In competition experiments, mutant (Nal^{r}, Kan^{r}) and wild-type bacteria (Nal^{r}) were grown for 48 h in 2YT, then equal amounts of bacteria (about 2.5×10⁵ each in 100 μl of 0.9% NaCl) were mixed and injected i.p. to mice. Dilutions of the infecting doses were plated on 2YT and 2YT Kan to estimate the ratio of mutant to wild-type bacteria in the inoculum. Mice were sacrificed after S days, and the spleen recovered and homogenised. To determine the proportion of mutant to wild-type, dilutions of the spleen homogenate were plated on 2YT and 2YT Kan. The competitive index (CI) was calculated as the proportion of mutant to wild-type bacteria recovered from the animals, divided by the proportion of mutant to wild-type in the inoculum. For in vitro growth assays, 5 ml of 2YT was inoculated with the infection dose. The cultures were incubated at 37° C. for 48 h with shaking (200 r.p.m.) and serial dilutions of the culture were plated to media with or without Kan. These experiments were performed in duplicate.

Infection of Macrophages and HeLa Cells.

Brucella spp can replicate within host cells. To determine if any of the mutants were defective in this property, subconfluent monolayers (2×10⁴) of murine J774 macrophages or human HeLa cells were inoculated with bacteria diluted to 6×10⁶ CFU ml⁻¹ in cell culture medium. Plates were centrifuged for 10 min at 1000 r.p.m. at room temperature and placed in a 5% CO₂ atmosphere at 370C. After 1 h, wells were washed three times and incubated for 48 h with cell culture medium supplemented with 50 μg ml⁻¹ gentamycin. At the end of the infection time, the monolayers were washed 3 times with cell culture medium and treated for 20 min with 200 μl of 0.1% Triton X-100 (Roche) in PBS. Serial dilutions of the lysates were plated onto 2YT plates for determination of CFU. Each infection was performed in triplicate.

The following Examples illustrate the invention.

### EXAMPLE 1

A first mutant was identified and the nucleotide sequence immediately following the transposon insertion was cloned. The sequence is shown as SEQ ID NO. 1. A translation from this sequence is shown as SEQ ID NO. 2.

The nucleotide sequence shows 59.4% identity from nucleotide 1-430 to the cysI gene of Pseudomonas aeruginosa at nucleotides 1109-1539 of the latter (EMBL accession number AF026066). The amino acid sequence shows 49.5% identity from amino acid 3-103 to amino acids 360-460 of the Pseudomonas aeruginosa CysI.

This demonstrates that the disrupted gene is at least partially identical to the cysI gene of Pseudomonas aeruginosa. The CysI gene has a putative function as a sulphite reductase (TrEMBL accession number 031037).

In the test for attenuation of virulence, the mutated microorganism was shown to be attenuated with a competitive index (CI) of 0.000293 (mean CI from 2 mice).

As the cysI genes in Salmonella typhimurium and E. coli strain B are transcribed as part of an operon with the cysH gene, it is possible that this attenuation is due to a polar effect on a presumed cysH gene in Brucella melitensis.

### EXAMPLE 2

A second mutant was identified, and the nucleotide sequence immediately following the transposon insertion was cloned. The sequence is shown as SEQ ID NO. 3. A translation from this sequence is shown in SEQ ID NO. 4.

The nucleotide sequence shows 63.3% identity from nucleotide 1-392 to the mgtB gene of Salmonella typhimurium at nucleotides 3531-3922 of the latter (EMBL accession number M57715). The amino acid sequence shows 44.3% identity from amino acid 1-131 to amino acids 672-802 of the Salmonella typhimurium mgtB.

This demonstrates that the disrupted gene is at least partially identical to the mgtB gene of Salmonella typhimurium. This gene has a putative function as a magnesium transport ATPase (SwissProt accession number P22036).

The amino acid sequence also shows 39.8% identity from amino acid 1-118 to the MgtA protein of E. coli K12 (SwissProt accession number P39168) from amino acid 663-780 of the latter.

In the test for attenuation of virulence the mutated microorganism was shown to be attenuated with a competitive index (CI) of 0.00189 (mean CI from 2 mice).

The mutant was also tested for invasion of macrophages and HeLa cells, and was found to be attenuated in both.

### EXAMPLE 3

A further mutant was identified, and the nucleotide sequence immediately following the transposon insertion was cloned. The sequence is shown as SEQ ID NO. 5. A translation from this sequence is shown as SEQ ID NO. 6.

The nucleotide sequence shows 63.2% identity from nucleotide 1-109 to the y4oU gene of Rhizobium sp. strain NGR234 (EMBL accession number AE000089) at nucleotides 5503-5611 of the latter. The amino acid sequence shows 36.4% identity from amino acid 1-122 to amino acid 7-126 of the Rhizobium sp. Y4oU protein (SwissProt accession number P55606).

This demonstrates that the disrupted gene is at least partially identical to the y4oU gene of Rhizobium sp. strain NGR234.

In the test for attenuation of virulence, the mutated microorganism was shown to be attenuated with a competitive index (CI) of 0.00362 (mean CI from 2 mice).

As the gene is potentially transcribed as part of an operon with the genes y4oV and y4oW, it is possible that this attenuation is due to a polar effect on presumed y4oV and/or y4oW genes.

The mutant was also tested for growth in macrophages and found to be attenuated.

### EXAMPLE 4

A further mutant was identified, and the nucleotide sequence immediately following the transposon insertion was cloned. The sequence is shown as SEQ ID NO. 7. A translation from this sequence is shown as SEQ ID NO. 8.

The nucleotide sequence shows 72.9% identity from nucleotide 46-300 to the cysK gene of Rhodobacter sphaeroides (EMBL accession number AF004296) at nucleotides 2969-3223 of the latter. The amino acid sequence shows 75.3% identity from amino acid 1-99 to amino acids 190-286 of the Rhodobacter sphaeroides CysK.

This demonstrates that the disrupted gene is at least partially identical to the cysK gene of Rhodobacter sphaeroides. This gene has a putative function as an O-acetylserine(thiol)lyase.

In the test for attenuation of virulence the mutated microorganism was shown to be attenuated with a competitive index (CI) of 0.000214 (mean CI from 2 mice).

The mutant was also attenuated in both macrophages and HeLa cells.

### EXAMPLE 5

A further mutant was identified, and the nucleotide sequence immediately following the transposon insertion was cloned. The sequence is shown as SEQ ID NO. 9. A translation from this sequence is shown as SEQ ID NO. 10.

The nucleotide sequence shows 69.4% identity from nucleotide 1-98 and 59.0% identity from nucleotides 315-375 to the meth gene of E. coli at nucleotides 4013-4110 and 3738-3798 of the latter (EMBL accession number AE000475). The amino acid sequence shows 37.7% identity from amino acid 11-79 to amino acids 854-922 of the E. coli MetH.

This demonstrates that the disrupted gene is at least partially identical to the metH gene of E. coli K12. This gene has a putative function as a homocysteine-NS-methyltetrahydrofolate transmethylase.

The amino acid sequence also shows 34.4% identity from amino acid 11-74 to the human 5-methyltetra-hydrofolate homocysteine methyltransferase (SwissProt accession number Q99707) from amino acids 881-944 of the latter.

In the test for attenuation of virulence the mutated microorganism was shown to be attenuated with a competitive index (CI) of 0.000225 (mean CI from 2 mice).

The mutant was also attenuated in both macrophages and HeLa cells.

### EXAMPLE 6

A further mutant was identified, and the nucleotide sequence immediately following the transposon insertion was cloned. The sequence is shown as SEQ ID NO. 11. A translation of this sequence is shown as SEQ ID NO. 12.

This nucleotide sequence shows 67.3% identity from nucleotide 1-101 to the ygjG gene of E. coli K12 (EMBL accession number U188997) at nucleotides 32481-32581 of the latter. The amino acid sequence shows 75% identity from amino acid 1-32 to amino acid 236-267 of the E. coli yqjG (SwissProt accession number P42620).

This demonstrates that the disrupted gene is at least partially identical to the ygjG gene of E. coli K12.

The amino acid sequence also shows 87.9% identity from amino acid 1-33 to amino acid 235-267 of an hypothetical protein (dbj) from Synechocystis sp. strain PCC6803 (TrEMBL accession number P74752).

In the test for attenuation of virulence the mutated microorganism was shown to be attenuated with a competitive index (CI) of 0,00109 (mean CI from 2 mice).

The mutant was also attenuated in macrophages and HeLa cells.

### EXAMPLE 7

A further mutant was identified, and the nucleotide sequence immediately following the transposon insertion was cloned. The sequence is shown as SEQ ID NO. 13. A translation from this sequence is shown as SEQ ID NO. 14.

This nucleotide sequences shows 54.5% identity from nucleotide 278-545 to the dnaJ gene of Thermus thermophilus (EMBL accession number L57504) at nucleotide 3072-3329 of the latter. The amino acid sequence shows 41.5% identity from amino acid 1-132 to amino acid 85-209 of the Thermus thermophilus DnaJ.

This demonstrates that the disrupted gene is at least partially identical to the dnaJ gene of Thermus thermophilus. This gene has a putative role as a chaparone (SwissProt accession number Q56237).

The amino acid sequence also shows homology to a number of other proteins belonging to the protein family of DnaJ proteins (accession number PF00226). This family includes the DnaJ protein from Salmonella typhimurium (SwissProt accession number Q60004) and of Mycobacterium leprae (SwissProt accession number Q02605).

In the test for attenuation of virulence the mutated microorganism was shown to be attenuated with a competitive index (CI) of 0.00004 (mean CI from 2 mice).

The mutant was also attenuated in macrophages and HeLa cells.

### EXAMPLE 8

A further mutant was identified, and the nucleotide sequence immediately following the transposon insertion was cloned. The sequence is shown as SEQ ID NO. 15. A translation of this sequence from nucleotides 1 to 537 is shown as SEQ ID NO. 16.

This is a previously unknown gene and has been termed bru1.

In the test for attenuation of virulence the mutated microorganism was shown to be attenuated with a competitive index (CI) of 0.000471 (mean CI from 2 mice).

The mutant was also attenuated in macrophages and HeLa cells.

### EXAMPLE 9

A further mutant was identified, and the nucleotide sequence immediately following the transposon insertion was cloned. The sequence is shown as SEQ ID NO. 17. A translation from this sequence is shown as SEQ ID NO. 18.

The gene shows 100% identity to nucleotides 1372-1491 of a sequence from Brucella abortus (AF011895). This sequence contains the ccrM gene known to encode an adenine DNA methyl transferase. However, the region of AF011895 with homology to the gene is located downstream of the ccrM gene, and no open reading frame has currently been ascribed to this region. This shows that Brucella abortus also contains a gene that is at least partially identical to the gene of B. melitensis.

In the test for attenuation of virulence the mutated microorganism was shown to be attenuated with a competitive index (CI) of 0.0000555 (mean CI from 2 mice).

The mutant was also attenuated in macrophages and HeLa cells.

### EXAMPLE 10

A further mutant was identified, and the nucleotide sequence on either side of the transposon insertion was cloned. The sequence at one end is shown as SEQ ID NO. 19, and the sequence at the other end is shown as SEQ ID NO. 21.

A translation from SEQ ID NO. 19 is shown as SEQ ID NO. 20. A translation from SEQ ID NO. 21 is shown as SEQ ID NO. 22.

The 641 nucleotide sequence (SEQ ID NO. 19) shows 57.8% identity from nucleotide 11-641 to the flhS gene of Paracoccus denitrificans (EMBL accession number AJ223460) at nucleotides 690-1308 of the latter. The 403 nucleotide sequence (SEQ ID NO. 21) shows 61.1% identity from nucleotide 59-233 to the flhR gene of Paracoccus denitrificans (EMBL accession number AJ223460). The 213 amino acid sequence (SEQ ID NO. 20) shows 36.8% identity from amino acid 1-210 to amino acid 221-426 of the Paracoccus denitrificans FlhS protein (TrEMBL accession number 054012). The 89 amino acid sequence (SEQ ID NO. 22) shows 44.6% identity from amino acid 18-82 to amino acid 166-230 of the Paracoccus denitrificans FlhR (TrEMBL accession number 054014).

This demonstrates that the disrupted genes are at least partially identical to the flhs and flhR genes of Paracoccus denitrificans.

The 213 amino acid sequence also shows 33.3% identity from amino acid 1-210 to amino acid 400-613 of the VsrB protein of Pseudomonas solanacearum (TrEMBL accession number Q52582).

The 89 amino acid sequence also shows 56.1% identity from amino acid 25-81 to amino acid 157-213 the VsrC protein of Pseudomonas solanacearum (TrEMBL accession number Q45415).

As the flhS and flhR genes of Paracoccus denitrificans are potentially transcribed as part of an operon with a putative open reading frame orf2, it is possible that this attenuation is due to a polar effect on a presumed gene homologous to orf2 or the actual insertion of the mini-Tn5 transposon in a presumed orf2.

In the test for attenuation of virulence the mutated microorganism was shown to be attenuated with a competitive index (CI) of 0.00052 (mean CI from 2 mice).

The mutant was also attenuated in macrophages and HeLa cells.

### EXAMPLE 11

A further mutant was identified, and the nucleotide sequence either side of the transposon insertion was cloned. The sequence at one end is shown as SEQ ID NO. 23, and the sequence at the other end is shown as SEQ ID NO. 25.

A translation from SEQ ID NO. 23 is shown as SEQ ID NO. 24. This predicted protein shows 66% identity to the Rbsc-2 protein of A. fulgodus at amino acids 27 to 44.

A translation from SEQ ID NO. 25 is shown as SEQ ID NO. 26. This sequence shows 56.3% identity to the rbsc-2 gene of A. fulgodus at nucleotides 2873 to 3007 and 37% identity to amino acids 96 to 149.

This demonstrates that the disrupted gene is at least partially identical to the rsbc2 gene of A. fulgidus (EMBL accession number AJ224684). The rbsc2 gene encodes a probable ribose ABC transporter permease protein.

In the test for attenuation of virulence, the mutated microorganisms was shown to be attenuated with a competitive index (CI) of 0.00638 (mean CI from 2 mice).

The mutant was also attenuated in macrophages and HeLa cells.

### EXAMPLE 12

A further mutant was identified, and the nucleotide sequence either side of the transposon insertion was cloned. The sequence at one end is shown as SEQ ID NO. 27, and the sequence at the other end is shown as SEQ ID NO. 29.

A translation of SEQ ID NO. 27 is shown as SEQ ID NO. 28. A translation of SEQ ID NO. 29 is shown as SEQ ID NO. 30.

The 360 nucleotide sequence (SEQ ID NO. 27) shows 54.8% identity from nucleotide 17-359 to the ugpA gene of E. coli K12 (EMBL accession number X13141) at nucleotides 1823-2165 of the latter. The 375 nucleotide sequence (SEQ ID NO. 29) shows 61.4% identity from nucleotide 62-372 to the ugpB gene of E. coli K12 at nucleotides 1321-1631 of the latter. The 119 amino acid sequence (SEQ ID NO. 28) shows 43.6% identity from amino acid 1-117 to amino acid 19-135 of the UgpA protein of E. coli K12 (SwissProt accession number P10905). The 112 amino acid sequence (SEQ ID NO. 30) shows 48.1% identity from amino acid 1-117 to amino acid 19-135 of the UgpA protein of E. coli K12 (SwissProt accession number P10904).

This demonstrates that the disrupted gene is at least partially identical to the ugp operon of E. coli K12.

As the ugpA and ugpB genes are transcribed as part of an operon with the ugpC and ugpE gene, it is possible that this attenuation is due to a polar effect on a presumed ugpC or/and ugpE gene in Brucella melitensis.

The mutant was also attenuated in macrophages and HeLa cells.

### EXAMPLE 13

A further mutant was identified, and the nucleotide sequence following the transposon insertion was cloned. The nucleotide sequence is shown as SEQ ID NO. 31.

The sequence shows 80% homology to the mtgtA gene of H. influenzae.

In the test for attenuation of virulence, the mutant was shown to be attenuated with a competitive index (CI) of 0.0002 (mean CI from 2 mice).

The mutant was also attenuated in macrophages and HeLa cells.

### EXAMPLE 14

A further mutant was identified, and the nucleotide sequence following the transposon insertion was cloned. The nucleotide sequence is shown as SEQ ID NO. 32.

The sequence shows 40% homology to a gene of unknown function in S. meliloti.

In the test for attenuation of virulence, the mutant was shown to be attenuated with a competitive index of 0.0000373 (mean CI from 2 mice).

The mutant was also attenuated in macrophages and HeLa cells.

### EXAMPLE 15

A further mutant was identified, and the nucleotide sequence following the transposon insertion was cloned. The nucleotide sequence is shown as SEQ ID NO. 33.

The sequence has no homologies to other known genes.

In the test for attenuation of virulence, the mutant was shown to be attenuated with a competitive index of 0.000277 (mean CI from 2 mice).

The mutant was also attenuated in macrophages and HeLa cells.

We claim:

1. A composition of matter comprising:
(a) a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(b) a polynucleotide encoding a peptide, wherein said peptide can be encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(c) a host transformed to express a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(d) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(e) a therapeutic or diagnostic composition comprising an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(f) a vaccine comprising a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or the means for expressing said peptide; or
(g) a vaccine comprising an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or
(h) an antibody, raised against: 1) a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof or, 2) a polynucleotide encoding a peptide, wherein said peptide can be encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof.

2. The composition of matter according to claim 1, wherein the composition comprises the peptide of (a), and wherein the homologue has at least 40% sequence similarity to the corresponding B. melitensis sequence.

3. The composition of matter according to claim 2, wherein the homologue has at least 60% sequence similarity.

4. The composition of matter according to claim 2, wherein the homologue has at least 90% sequence similarity.

5. The composition of matter according to claim 1, wherein the composition comprises the peptide of (a), and wherein the peptide comprises an amino acid sequence selected from the group consisting of SEQ ID NOS. 2, 4, 6, 8, 10, 12, 14, 16, 18, 20, 22, 24, 26, 28 and 30.

6. The composition of matter according to claim 1, wherein the composition comprises the polynucleotide of (b), and wherein the polynucleotide encodes a peptide comprising an amino acid sequence selected from the group consisting of SEQ ID NOS. 2, 4, 6, 8, 10, 12, 14, 16, 18, 20, 22, 24, 26, 28 and 30.

7. The composition of matter according to claim 1, wherein the composition of matter comprises the polynucleotide of (b), and wherein the homologue has at least 40% sequence similarity to the corresponding B. melitensis sequence.

8. The composition of matter according to claim 7, wherein the homologue has at least 60% sequence similarity.

9. The composition of matter according to claim 7, wherein the homologue has at least 90% sequence similarity.

10. The composition of matter according to claim 1, wherein the composition of matter comprises the polynucleotide of (b), and wherein the polynucleotide comprises a sequence selected from the group consisting of SEQ ID NOS. 1, 3, 5, 7, 9, 11, 13, 15, 17, 19, 21, 23, 25, 27, 29, 31, 21 and 33.

11. The composition of matter according to claim 1, wherein the composition of matter comprises the attenuated microorganism of (d), and wherein the mutation is insertional inactivation or a gene deletion.

12. The composition of matter according to claim 1, wherein the composition of matter comprises the attenuated microorganism of (d), and wherein the microorganism is a Brucella species.

13. The composition of matter according to claim 1, wherein the composition of matter comprises the therapeutic or diagnostic composition of (e), and wherein the microorganism comprises a mutation in a further nucleotide sequence.

14. The composition of matter according to claim 1, wherein the composition of matter comprises the therapeutic or diagnostic composition of (e), and wherein the microorganism comprises a heterologous antigen, therapeutic peptide or nucleic acid.

15. A method for screening potential drugs or for the detection of virulence wherein said method comprises the use of at least one of the following: (a) a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; or (b) a polynucleotide encoding a peptide, wherein said peptide can be encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof.

16. A method for the treatment or prevention of a condition associated with infection by Gram-negative bacteria wherein said method comprises administering to a patient in need of such treatment or prevention one or more of the following:
(a) a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof;
(b) a polynucleotide encoding a peptide, wherein said peptide can be encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof; and
(c) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof.

17. The method according to claim 16, wherein the condition is Brucellosis.

18. A method for screening for the identification of an antimicrobial drug wherein said method comprises using in a screening assay a peptide encoded by a nucleotide sequence selected from the group consisting of SEQ ID NOS. 1, 3, 5, 7, 9, 11, 13, 15, 17, 19, 21, 23, 25, 27, 29, 31, 21 and 33 of B. melitensis, homologues thereof from Gram-negative bacteria, and functional fragments thereof.

19. A peptide comprising an amino acid sequence selected from the group consisting of SEQ ID NOS. 2, 4, 6, 8, 10, 12, 14, 16, 18, 20, 22, 24, 26, 28 and 30.

20. The peptide according to claim 19, wherein the peptide comprises the amino acid sequence of SEQ ID NO:10.
